(19) United States
(12) Patent Application Publication
(10) Pub. No.: US 2025/0049347 A1
(43) Pub. Date: Feb. 13, 2025

(54) NEURAL NETWORK TO PREDICT KNEE MEDIAL JOINT CONTACT FORCE FROM CUSTOM INSTRUMENTED INSOLE

(71) Applicant: UNIVERSITY OF MARYLAND, COLLEGE PARK, College Park, MD (US)

(72) Inventors: Jae Kun SHIM, Clarksville, MD (US); Ross MILLER, Bethesda, MD (US); Samantha SNYDER, Columbia, MD (US)

(21) Appl. No.: 18/797,404

(22) Filed: Aug. 7, 2024

Related U.S. Application Data
(60) Provisional application No. 63/518,102, filed on Aug. 7, 2023.

(51) Int. Cl. A61B 5/11; G01L 5/00; G06F 3/01

(52) U.S. Cl. CPC A61B 5/112; A61B 5/1123; G01L 5/0038; G06F 3/011

(57) ABSTRACT

Methods and systems are provided for determining medial joint contact force. For example, methods and systems described herein may train a neural network model to determine medial joint contact force. A plurality of training data is obtained and a plurality of heel strike to toe-off time periods are extracted from the training data. A gait categorization label is assigned. A ground reaction force and joint contact forces are determined. An input training array and an output training array is provided to the neural network. The neural network is trained to output a predicted joint contact force value corresponding to a calculated joint contact force measured from force plate data, based only on insole data. The trained neural network model is stored after it has been validated.

### CROSS-REFERENCE TO RELATED APPLICATION(S)

The present application is based on, claims priority to, and incorporates herein by reference in its entirety for all purposes, U.S. Provisional Patent Application Ser. No. 63/518,102, filed Aug. 7, 2023.

### STATEMENT OF GOVERNMENT SUPPORT

N/A

### BACKGROUND

Medial tibiofemoral joint reaction force is a clinically relevant variable for knee osteoarthritis progression and can currently only be estimated using complex musculoskeletal models developed from expensive, complex, and highly sensitive laboratory equipment. Musculoskeletal model estimation of this variable is time-consuming, expensive, requires trained researchers, and is restricted to lab settings. To date, no estimation of medial tibiofemoral joint reaction force can be made in real world/real time scenarios.

However, it would be extremely useful to have a low cost, low complexity method and system for prediction of such forces that are relevant to osteology, arthology, or orthopaedic treatment. For example, knee osteoarthritis typically occurs on the medial aspect of the knee, so frequent and greater stress on this area of the knee is of particular interest. Larger knee adduction moment (KAM), a surrogate measure for medial knee force, is associated with greater risk and development of knee osteoarthritis. Although there is a relationship between KAM and medial joint contact force (MJCF), the correlation between the two variables varies, and reducing KAM does not necessarily reduce MJCF.

Furthermore, many individuals who develop osteoarthritis or similar conditions do not seek care or diagnostic analysis before these conditions have already developed, in many cases severely. Thus, having a simple, low-cost, and everyday (outside-lab) system for monitoring joint forces can allow for earlier prediction and diagnosis of problems like knee osteoarthritis.

### SUMMARY

The following presents a simplified summary of one or more aspects of the present disclosure, in order to provide a basic understanding of such aspects. This summary is not an extensive overview of all contemplated features of the disclosure, and is intended neither to identify key or critical elements of all aspects of the disclosure nor to delineate the scope of any or all aspects of the disclosure. Its sole purpose is to present some concepts of one or more aspects of the disclosure in a simplified form as a prelude to the more detailed description that is presented later.

In some aspects, the present disclosure may provide a method for training a neural network model to determine medial joint contact force. A plurality of training data can be obtained. The plurality of training data can include a set of corresponding force plate data and motion capture data. The data of each set can be acquired simultaneously. A plurality of heel strike to toe-off time periods from the plurality of training data can be extracted. A gait categorization label corresponding to the plurality of training data can be assigned. A ground reaction force, a plurality of knee moments, and a plurality of knee angles can be determined based on said force plate data and motion capture data. A plurality of joint contact forces can be determined at a plurality of time points during the plurality of heel strike to toe-off time periods. An output training array can be provided to the neural network. The output training array can include the plurality of joint contact forces corresponding to the plurality of heel strike to toe-off periods. The neural network model can be trained to output a predicted joint contact force value. The predicted joint contact force value can correspond to a calculated joint contact force measurement from force plate data and based only on insole data. The trained neural network model can be stored after it has been validated.

In some aspects, the present disclosure may provide a system for determining medial joint contact force of a user. The system can include an insole, a processor in communication with the insole, and a memory in communication with the processor. The insole can include at least one insole sensor configured to measure a force associated with a subject's gait and a wireless transmitter connected to the at least one insole sensor to transmit output data of the at least one insole sensor. The memory can have instructions stored thereon that, when executed, cause the processor to receive a plurality of output data from the at least one insole sensor. An activity category can be determined, corresponding to the plurality of data. A temporal bin of the data can be extracted, associated with a heel strike to toe-off period. The temporal bin of data and the corresponding activity category can be provided to a trained neural network model. A medial joint contact force occurring during the heel strike to toe-off period can be determined using the trained neural network model. A joint force summary can be generated. The joint force summary can be transmitted to the user.

These and other aspects of the disclosure will become more fully understood upon a review of the drawings and the detailed description, which follows. Other aspects, features, and embodiments of the present disclosure will become apparent to those skilled in the art, upon reviewing the following description of specific, example embodiments of the present disclosure in conjunction with the accompanying figures. While features of the present disclosure may be discussed relative to certain embodiments and figures below, all embodiments of the present disclosure can include one or more of the advantageous features discussed herein. In other words, while one or more embodiments may be discussed as having certain advantageous features, one or more of such features may also be used in accordance with the various embodiments of the disclosure discussed herein. Similarly, while example embodiments may be discussed below as devices, systems, or methods embodiments it should be understood that such example embodiments can be implemented in various devices, systems, and methods.

### BRIEF DESCRIPTION OF THE DRAWINGS

FIG. 1 is a block diagram conceptually illustrating a system of determining medial joint contact force according to some embodiments.

FIG. 2 is a flow diagram illustrating an example process for determining medial joint contact force.

FIG. 3 is a flow diagram illustrating an example process for training a neural network model using force place and motion capture data.

FIG. 4 illustrates example marker locations on a participant, according to some embodiments.

FIG. 5 illustrates of overview of data collection, preparation, and analysis steps, according to some embodiments.

FIG. 6 illustrates average prediction and musculoskeletal models calculated for example walking and running neural network models, according to some embodiments.

### DETAILED DESCRIPTION

The detailed description set forth below in connection with the appended drawings is intended as a description of various configurations and is not intended to represent the only configurations in which the subject matter described herein may be practiced. The detailed description includes specific details to provide a thorough understanding of various embodiments of the present disclosure. However, it will be apparent to those skilled in the art that the various features, concepts and embodiments described herein may be implemented and practiced without these specific details. In some instances, well-known structures and components are shown in block diagram form to avoid obscuring such concepts.

Measuring MJCF during activities that greatly load the knee can be a better indicator of the magnitude of mechanical loads on the part of the knee where knee osteoarthritis most often occurs. In knee osteoarthritis patients, greater peak MJCF during walking has been associated with medial tibial cartilage volume loss and knee osteoarthritis progression. Because of the relationship between larger MJCF and knee osteoarthritis risk, MJCF is a useful factor in knee osteoarthritis research. However, the MJCF is a difficult variable to measure. These in-vivo medial knee forces can be accurately measured through a force-sensing knee replacement implant, but before individuals undergo knee arthroplasty, this tool is not feasible for broad use. Therefore, it is common to use less invasive methods using musculoskeletal modeling techniques to estimate MJCF from ground reaction force and motion capture data. These model based techniques can measure peak MJCF during walking with minimum detectable changes of 0.25 BW.

Measuring MJCF has historically been restricted to a gait analysis laboratory, which is time-consuming and requires expensive equipment. To simplify gait analysis measurements without compromising accuracy, researchers have used artificial neural networks and wearable sensors. Despite overall success in applying wearable sensor data to neural networks to predict biomechanical variables, there has been minimal research estimating knee joint contact forces using neural networks and wearables. When estimating total knee joint contact force, neural networks were combined with variables obtained through typical gait analysis data collection or inertial measurement units. There is little research estimating MJCF with wearables and neural networks or only data from instrumented shoe insoles. Instrumented insoles have been used in the past to estimate variables relevant to MJCF with high accuracy such as ground reaction forces, joint angles, KAM, and total knee joint contact force. Insoles are attractive for this purpose due to their potential for portable applications in daily living.

Therefore, the systems and methods described herein aim to estimate MJCF during walking and running with instrumented insoles and deep learning methods. Because women are more likely to be affected by knee osteoarthritis, the systems and methods described herein aim to estimate this biomechanical risk factor in women.

### Example Hardware Systems

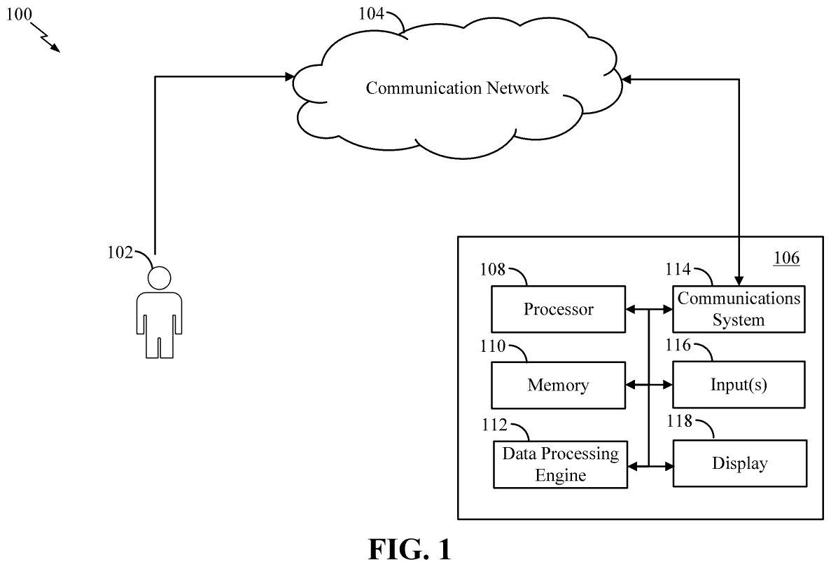

Certain techniques and advantages described herein can be achieved via a variety of different hardware configurations. For example, software instructions that operate on force data, or motion capture data, from a sensor could operate on a processor of the same device as the sensor, a locally connected device, or a remote resource. Thus, FIG. 1 below provide general examples of possible configurations of hardware implementing aspects of the disclosure.

FIG. 1 shows a block diagram illustrating an example of a system 100 for determining medial joint contact force using data from instrumented insoles. In some examples, a computing device 106 can obtain data from a user sensor 102 (such as an instrumented insole or motion capture senor(s)) or other connected device via a communication network 104. For example, the user sensor 102 may cover the insole described in, hereby incorporated herein in its entirety for all purposes, U.S. Provisional Patent Application Ser. No. 63/518,102, filed Aug. 8, 2023. In some examples, the data of the user sensor 102 can include a plurality of data points corresponding to a user's steps, a 3D model of the user's motion, an image, a video frame, a numerical reading, or any other suitable data.

As will be understood from the description herein, the user sensor 102 may be a standalone sensor, or may be a variety of types of sensors. For example, user sensor 102 may be an insole sensor and/or one or more piezoresistive force sensors. In some examples, the user sensor 102 may include one or more sensors in an insole of a user's shoe, within a specialized sock, and/or a specialized article of footwear. For example, one or more sensors may be placed in the heel region, the ball or metatarsal region, and/or the forefoot region of a user's foot. The one or more sensors may be spatially dispersed throughout the bottom of a user's foot. In some examples, the user sensor 102 can include 3 axes, or any of number of axes. In examples where multiple sensors are used for the user sensor 102, the sensors may be of the same or different sensor types. In some examples, the insole which the user sensor 102 is located can have its own power supply (i.e., battery), processor, and/or wireless transmitter.

The computing device 106 can include a processor 108. In some embodiments, the processor 308 can be any suitable hardware processor or combination of processors, such as a central processing unit (CPU), a graphics processing unit (GPU), an application specific integrated circuit (ASIC), a field-programmable gate array (FPGA), a digital signal processor (DSP), a microcontroller (MCU), cloud resource, etc. In some examples, the computing device 106 can be the user's mobile phone, smartwatch, wearable device, or other similar device.

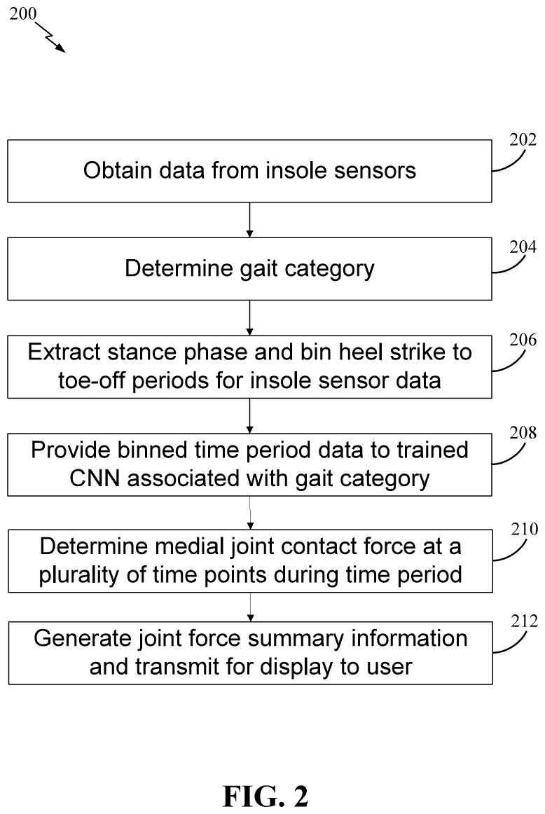

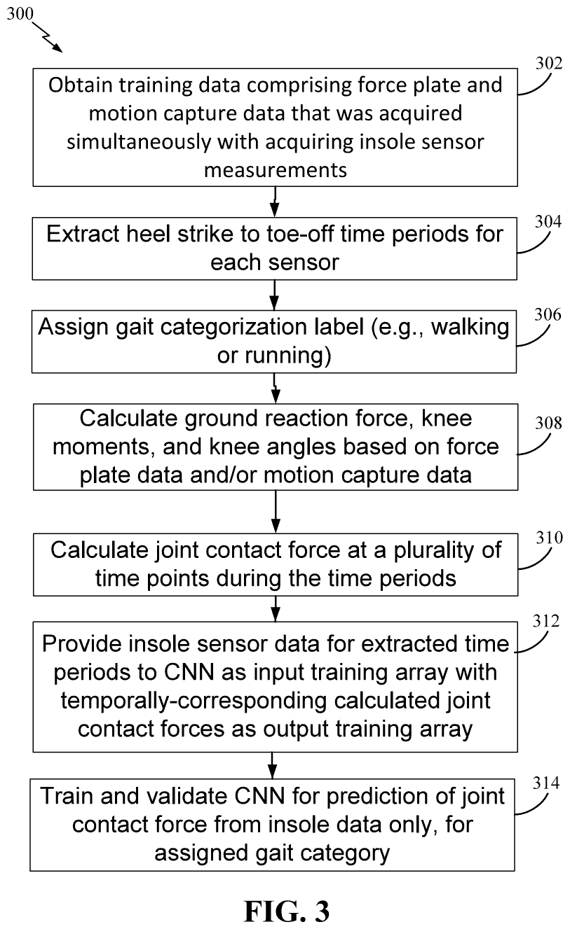

The computing device 106 can further include, or be connected to, a memory 110. The memory 110 can include or comprise any suitable storage device(s) that can be used to store suitable data (e.g., force data, motion capture data, etc.) and instructions that can be used, for example, by the processor 108. Methods for determining medial joint contact force using the data of user sensor 102 may operate as independent processes/modules, such as a separate data processing engine 112 that runs on the same processor 108 or a specialty processor (such as a GPU) that achieves greater efficiency in processing the data using a neural network, as described below. In some embodiments, the data processing engine 112 may be a hardware or virtual processing resource (e.g., a GPU) for efficient performance of some or all of processes 200 and 300, described below. For example, the data processing engine 112 may be a GPU that performs the neural network model described above with respect to FIGS. 2 and 3. In some examples, the data processing engine 112 can store a neural network model. For example, the data processing engine 112 can store the CNN model(s) described in more detail below, a set of CNN model tailed to the user, a general set of CNN model, and/or other types of neural network models. In some examples, the model(s) may be stored in a remote resource, such as the cloud. The memory 110 can include any suitable volatile memory, non-volatile memory, storage, or any suitable combination thereof. For example, memory 110 can include random access memory (RAM), read-only memory (ROM), electronically-erasable programmable read-only memory (EEPROM), one or more flash drives, one or more hard disks, one or more solid state drives, one or more optical drives, etc.

In further examples, computing device 106 can receive or transmit information and/or any other suitable system over a communication network 104. In some examples, the communication network 104 can be any suitable communication network or combination of communication networks. For example, the communication network 104 can include a Wi-Fi network (which can include one or more wireless routers, one or more switches, etc.), a peer-to-peer network (e.g., a Bluetooth network), a cellular network (e.g., a 3G network, a 4G network, a 5G network, etc., complying with any suitable standard, such as CDMA, GSM, LTE, LTE Advanced, NR, etc.), a wired network, etc. In one embodiment, communication network 104 can be a local area network, a wide area network, a public network (e.g., the Internet), a private or semi-private network (e.g., a corporate or university intranet), any other suitable type of network, or any suitable combination of networks. Communications links shown in FIG. 1 can each be any suitable communications link or combination of communications links, such as wired links, fiber optic links, Wi-Fi links, Bluetooth links, cellular links, etc. In some examples, the communication network 104 can include two types of transmission: (1) a transmission between the user sensor 102 and a mobile device, and (2) transmission between a mobile device to a remote device and/or the cloud.

In further examples, computing device 106 can further include one or more input(s) 116 and/or one or more output(s) 118. In one embodiment, the output 118 can be any suitable output devices, such as a screen, a computer monitor, a touchscreen, a television, an infotainment screen, a speaker, an earphone, a mobile phone, a watch etc. to display the report. In further embodiments, the output 118 can deliver the report through any suitable media, such as via sound, alarm, or notifications, played or displayed by the output device on the computing device 106 itself, or on other peripheral devices (e.g. a mobile phone or a watch) wired to or wirelessly connected to device 106. In further embodiments, and/or the input(s) 116 can include any suitable input devices (e.g., a keyboard, a mouse, a touchscreen, a microphone, etc.). In yet further embodiments, the user sensor 102 may be a camera that exports motion capture data to a remote resource 106, then receives force determinations from the resource 106 and displays them on a display 118.

### Example Medial Joint Contact Force Determination Process

FIG. 2 is a flow diagram illustrating an example process 200 for determining medial joint contact force, in accordance with some aspects of the present disclosure. As described below, a particular implementation can omit some or all illustrated features/steps, may be implemented in some embodiments in a different order, and may not require some illustrated features to implement all embodiments. In some examples, an apparatus (e.g., computing device, processor with memory, etc.) can be used to perform example process 200. However, it should be appreciated that any suitable apparatus or means for carrying out the operations or features described below may perform process 200.

At step 202, data from one or more insole sensors is obtained. In some examples, the insole sensors may be embedded into an insole of a shoe. The sensor may include piezoresistive force sensors, though other types of sensors are also contemplated such as compressive sensors (like strain gauges), accelerometers, gyroscopes, piezoelectric sensors, inertial sensors, and combinations thereof. In some examples, the data may be obtained from other types of wearable sensors, including sensors not embedded in an insole of a shoe but otherwise worn on, under, or about the foot. Moreover, the number of sensors may vary depending on their type and how they are situated relative to the foot. As described below, the inventors have determined that a set of multiple sensors disposed under a user's foot, in which at least one sensor is at/near the heel, at least one sensor is at/near the metatarsal region of the foot, and at least another sensor is at/near the toes is a beneficial arrangement (though in some cases it may be sufficient to have sensors only at the heel and toes, or only at the ball of the foot, etc.). In some examples, data corresponding to the individual(s) wearing or using the sensor(s) may be combined with the sensor data (e.g., age, height, weight, gender, etc.).

At step 204, a gait category is determined. In some examples, an activity category may be determined at step 204, in place of or in addition to a gait category. For example, the data obtained from the insole sensor at step 202 may correspond to one or more gate cycles (such as a complete footfall, whether the subject's heel, ball, etc. touches the ground first) of an individual walking and/or running. The gait categorization may be performed automatically, according to user input, from speed measurements determined from GPS/location data, and/or inertial measurement readings extracted from the data. In some examples, the process 200 may also determine if the insole sensor data corresponds to any gait, or if the data is from another activity (e.g., biking, jumping, etc.). In other words, if data is acquired, but does not resemble walking or another gait category that can be evaluated by process 200, the data can be ignored. In other embodiments, different models may apply to running, walking, and other activities, and process 200 may automatically select the appropriate model based on characteristics of the sensor data.

At step 206, the process 200 extracts a stance phase and identifies, segregates, or otherwise ‘bins’ heel strike to toe-off periods for insole sensor data. For example, the stance phase may represent a period between a heel hitting the ground to the moment at or before the toe coming off of the ground, if the user is walking. For some subjects, their footfall may entail the ball or forefoot striking the ground first, such as when sprinting or wearing specialized gear (e.g., cleats, supportive running shows, etc.). Thus, the identification of a complete step/stance phase may vary by activity, and process 200 may utilize the gait category information to more accurately bin phases. In some examples, the extraction of the stance phase may depend on the gait determination, as a stance phase may differ between a running gait and a walking gait. In some examples, the process 200 may identify origins and midpoints of the obtained motion data, such as a pelvis origin, a knee joint, and/or other joint locations to extract the stance phase and bin heel strike to toc-off periods.

At step 208, the binned time period data is provided to a trained neural network, such as a trained convolutional neural network (CNN) associated with the determined gait category. In some examples, the bins may include stance cycles. In some examples, the stance cycles within the bins may also indicate the information specific to the corresponding individual (e.g., height, weight, gender, etc.), allowing the CNN to select a model.

At step 210, the process 300 determines a medial joint contact force at a plurality of time points during a time period. In some examples, the medial joint contact force may be determined by the output of the CNN. As described further below, a neural network may be configured to output various ways of measuring joint contact force, such as various units of measurement, total load, average load, etc.

At step 212, the process 300 generates joint force summary information and transmits the information to display to a user. In some examples, a user may want to minimize the force experienced by one or more of their joints during a given time period and/or activity. For example, the joint force summary may notify the user when a predetermined threshold has been reached. In other examples, a doctor or medical professional may use the joint force summary information to monitor a patient's joint. The information may be sent to the doctor or medical professional, who can recommend medication, supplements, exercises, stretches, etc. based on the summary. In some examples, the recommendations may be automatically generated without any interference by a doctor or medical professional. In some examples, athletes, athletic trainers, coaches, etc. may be provided with the joint force summary information to monitor information regarding the athlete's performance during a specific sport. In some examples, doctors, medical professionals, and/or researchers may recommend gait alterations based on the results provided by process 200 and the methods disclosed herein. In particular, gait alteration recommendations may be targeted at reducing MJCF, such as changes to trunk lean and toe progression angles.

### Example Neural Network Training Process

FIG. 3 is a flow diagram illustrating an example process for training a neural network model using force place and motion capture data. As described below, a particular implementation can omit some or all illustrated features/steps, may be implemented in some embodiments in a different order, and may not require some illustrated features to implement all embodiments. In some examples, an apparatus (e.g., computing device, processor with memory, etc.) can be used to perform example process 300. However, it should be appreciated that any suitable apparatus or means for carrying out the operations or features described below may perform process 300.

At step 302, the process 300 obtains training data. The training data can include force plate and motion capture data that was acquired simultaneously. The training data may be acquired by one or more insole sensor measurements. And, the training data may be predetermined to correspond to a specific gait category, such as walking or running, or some other activity.

At step 304, the process 300 extracts heel strike to toe-off time periods for each sensor (or other stance phase, if heel strike is not first, or toe-off is not last). For example, a threshold value may be set that can allow for labeling of each heel strike and toe off initiation. In some examples, the labels may be automatically generated by the systems and methods described herein, such as by monitoring a time series of force information for a sensor located near a heel and a time series of force information for a sensor located near toes, and determining a time window from the moment a threshold force is detected by the heel sensor, continuing through a threshold of force measured from the toc sensor, until the threshold force of the toe sensor is no longer met.

At step 306, the process 300 assigns a gait categorization label. For example, the training data obtained at step 302 may correspond to one or more cycles of an individual walking and/or running. The gait categorization may be assigned automatically, using visual analysis and/or inertial measurement readings extracted from the data. In some examples, the training data may include automatically generated labels corresponding to one or more cycles/periods of data corresponding to a walking and/or running gait category.

At step 308, the process 300 calculates a ground reaction force, a knee moments, and knee angles based on data from the force plate and/or the motion capture data. In some examples, the training data may identify origins and midpoints of the obtained motion data, such as a pelvis origin, a knee joint, and/or other joint locations to extract the stance phase and bin hell strike to toe-off periods. Methods for calculating these forces are known, as well as described below.

At step 310, the process 300 calculates a joint contact force at a plurality of time points during a plurality of time periods. In some examples, the time points may represent percentages/progressions of a stance phase.

At step 312, the process 300 provides the insole sensor data for the extracted time periods to a neural network (such as a CNN) as an input training array. The temporally-corresponding calculated joint contact forces corresponding to the extracted time periods may also be provided to the CNN as an output training array. In some examples, the input and output training arrays may be normalized to apply to any/all insole data, regardless of an individual's gender, weight, age, height, etc. In other examples, the in and output arrays may be specific to a specific group or groups of individuals.

At step 314, the process 300 trains and validates the CNN for prediction of joint contact force using only the insole data. In particular, the CNN may be a model assigned to the gait categorization label of step 306.

### EXAMPLE EMBODIMENTS AND EXPERIMENTS

The inventors performed certain experiments using the foregoing process, as described about with respect to process 200 of FIG. 2 and process 300 of FIG. 3. The following descriptions provides examples and data from those experiments. Accordingly, the following description is directed to non-limiting examples.

Participants: Nine young, female adults with no history of lower extremity injuries and no neurological or orthopedic disorders volunteered to participate in this study. Due to sensor malfunctioning for one participant during running, eight participants were used for running data (22.5±2.8 years old, 164.6±35.0 cm, 61.81±5.95 kg) and nine participants were used for walking data (23.7±3.5 years old; 163.7±4.4 cm; 61±6.5 kg). Five out of the eight running participants ran regularly (3.3±1.7 miles/week). Participants provided informed consent of protocol, which was approved by the University of Maryland at College Park Institutional Review Board (#1335286).

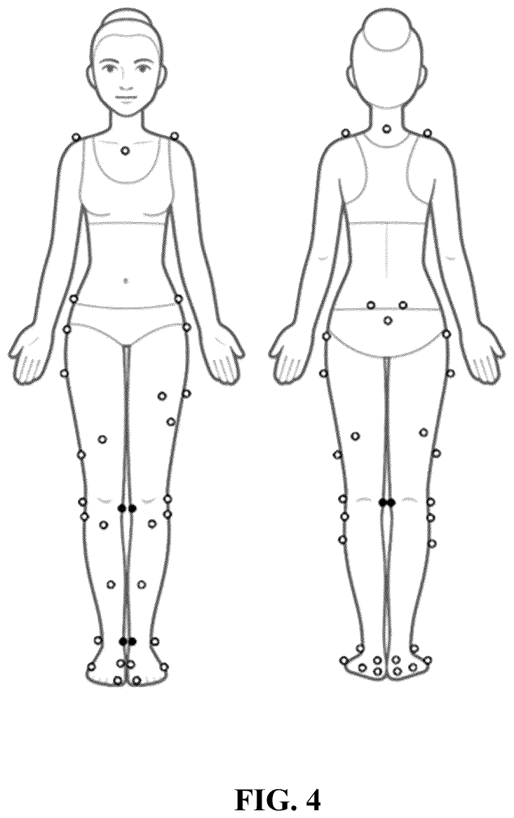

Experimental Setup and Procedures: Thirteen Vicon motion capture cameras (Vicon, Oxford, UK) captured marker positions at 200 Hz while ten six-degree-of-freedom force plates placed in a single row within a raised platform (Kistler, Switzerland) recorded ground reaction forces at 1000 Hz. All participants wore tight-fitting clothing, retroreflective markers (FIG. 4), and the same running shoes with the right foot instrumented with five tri-axial piezoresistive force sensors located under high-pressure areas of the foot. First, participants performed a static calibration trial where they stood still in an anatomical position on two force plates. Next, participants performed a maximum of ten minutes of walking and ten minutes of overground running where eight walking and eight running trials were captured. The participants and walking trials utilized in this analysis were from the same data set of the previous study. One trial was counted when the participant passed across the force plates. At the start of the walking and running trials participants put their right leg on a chair and the insole sensors were zeroed. For walking and running trials, the participant was randomly assigned to start at either a subjectively slow or fast self-selected speed. The participant then was instructed to increase or decrease their speed for subsequent trials.

Data Processing: For both walking and running, five trials where the right foot was in contact with one force plate were extracted. Markers were labeled in Vicon and filtered in Visual3D (C-Motion, Germantown, MD, USA) with a 4^{th} order dual-pass Butterworth filter with a frequency of 6 Hz. To filter the ground reaction force data, a 4th-order dual-pass Butterworth filter was also applied with a cutoff frequency of 50 Hz. Automatic gait detection was used in Visual3D to determine the stance phase, heel strike to toe-off, of each walking and running trial. From the calibration trial marker positions, a linked-segment model of each subject was created. The pelvis segment origin was defined as the mid-point between the middle of the anterior and posterior iliac spines. The knee joint center was defined between the medial and lateral femoral epicondyles and ankle joint between the malleoli markers, and later reconstructed as virtual joint centers based on marker positions in the calibration trials. Iterative Newton-Euler inverse dynamics was used to calculate the ankle, knee and hip moments, with the knee moment relative to the tibia reference frame. Joint angles were calculated using a Cardan XYZ rotation.

Inverse dynamics calculated variables, extracted from Visual3D, ankle, knee, and hip angles and moments along with ground reaction forces, were used to calculate MJCF using a reduction modeling approach in MATLAB 2021 (The Math Works, Natick, MA, USA). A reduction model approach is a common, albeit simplified, way to calculate medial knee joint forces. From data summarized in previous literature, muscle cross-sectional areas were calculated for hamstrings (bicep femoris, semitendinosus, and semimembranosus), gastrocnemius (lateral gastrocnemius and medial gastrocnemius), soleus, and gluteus maximus muscles. The gastrocnemius and soleus force were calculated based on ankle plantar flexion moments, and the hamstring and gluteus maximus muscles were calculated from hip extension moment. The relative contribution of each muscle was determined by cross-sectional areas. After accounting for hamstring and gastrocnemius contribution to the knee moment, the quadricep force was calculated. Moment arms were expressed as best fitting functions of hip, knee and ankle angles. except for the patellar moment arm, which was expressed as a quadratic function using average female value. Muscle orientations were expressed as quadratic functions of the knee flexion angle using average female values. Lateral collateral, medial collateral, anterior cruciate, and posterior cruciate ligaments were calculated based on methods in previous research using moment arms and orientations for anterior and posterior cruciate ligaments from results from Herzog and colleagues.

The total MJCF was calculated by taking the moment about the lateral aspect of the knee. For each subject, the model tibial plateau was linearly scaled to segment width based on an average female tibial plateau spanning 4.5 cm between contact points. Lastly, the reduction model calculated MJCF was scaled by body weight (BW). This calculation of MJCF considers co-contraction of muscles about the knee, which may be used to account for as there is an association between increased levels of co-contraction of knee muscles in individuals with more severe knee osteoarthritis. Furthermore, despite the mathematical simplifications, the applied method produces results reflective of walking and running data collected from participants with instrumented knee implants.

Each stance phase MJCF and insole sensor data was scaled to 101 time points representing 0 to 100% of the stance phase. Due to the relatively small sample size, an unequal number of trials were used from each participant to ensure the maximum amount of data possible. There was a total of 104 walking steps and 105 running steps.

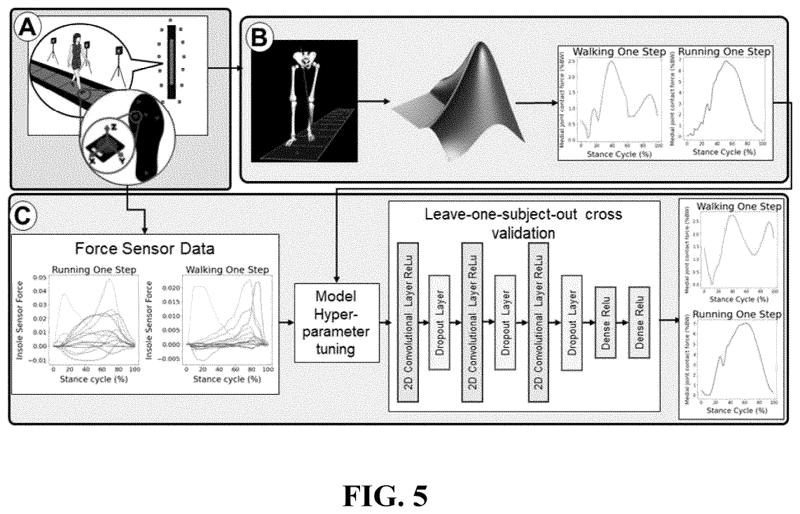

Data Analysis: A CNN outperformed a feed-forward neural network and recurrent neural network to predict KAMs using the instrumented insoles. Similarly, the systems and methods described herein are also predict a time series biomechanical variable describing the load on the knee, MJCF. A convolutional neural network (CNN) was selected. Two CNNs were developed for analysis. Running and walking models contained three 2D convolutional layers, each followed by dropout layers (rate=0.3 for walking, rate=0.1 for running). After the last dropout layer, there was a flatten layer and two dense layers (Panel C, FIG. 5). Two task specific models were created, one for walking and one for running, and developed in Python (version 3.8.3) using the TensorFlow library. Each model was input with 15 data points per 101 time steps from the instrumented insole sensors. With Keras Bayesian Optimization Tuner, each model with initially hyperparameter tuned to select ideal values for the learning rate, number of ReLu activated units in the convolutional layers. Based on the tuner results, the model parameters were applied in new models and trained on the data. Adam optimization algorithm was applied as the loss function and the models minimized mean squared error. The models were trained for 150 epochs, and to prevent overfitting, model training was stopped when the loss did not decrease for more than 15 epochs. For each model, it is recommended to apply leave-one-subject-out cross validation to account for the smaller sample size. This technique trains the model on all participants except for one participant who is used as test data to evaluate the model. Leave-one-subject-out cross validation was used previously in similar studies applying wearables and machine learning to relatively small sample sizes This process is iterated through for all participants, and the averages and standard deviations from the cross validated test sets were used for statistical analysis.

Statistical Analysis: The CNN model estimates of MJCF were compared to the reduction model approach calculated MJCF using Pearson's correlation coefficient, r, and mean squared error (MSE). A 95% confidence interval (CI) was calculated for each Pearson's correlation coefficient using a Fishers z-transform of the correlation coefficient. Percent differences between reduction model approach calculated peak values and CNN estimated peak values were calculated as well.

Results: After hyperparameter tuning, the walking best performing model included 160, 80, and 16 ReLu neurons in the first, second and third layers respectively. The running model contained 128, 48, and 176 ReLu neurons in the respective 2D convolutional layers.

The running model performed with greater correlation coefficients (0.98±0.03) compared to the walking model (0.98±0.03), while the walking model performed with lower MSE (0.06±0.10 BW) compared to the running model (0.41±0.72 BW) (Table 1).

Table 1 Walking and running model estimates compared to the musculoskeletal calculated medial knee joint contact force. One standard deviation is included for mean correlation coefficients and MSE.

Both the running and walking models underestimated peak MJCF (Table 2). The walking model had larger peak MJCF percentage differences (5.70±12.90%) compared to the running model (4.11±14.50%)

Table 2 Walking and running model peak estimates compared to the musculoskeletal calculated medial peak knee joint contact force.

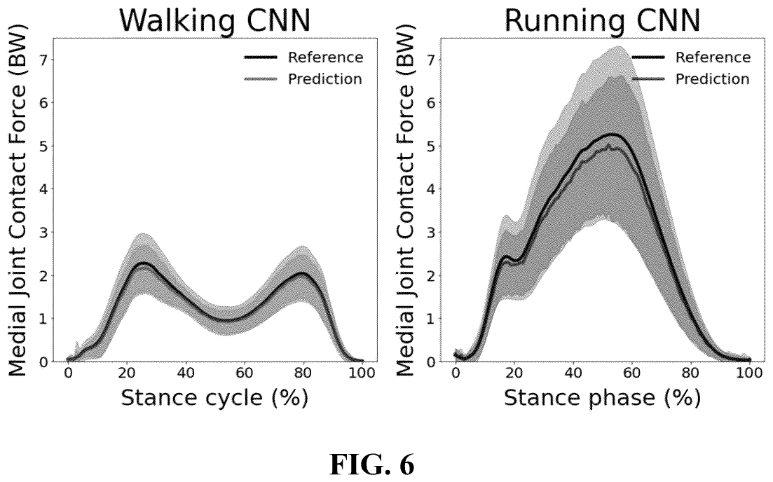

The mean waveforms of MJCF predicted by the CNN models and calculated by musculoskeletal modeling are shown for walking and running in FIG. 6.

Knee MJCF is one biomechanical risk factor for knee osteoarthritis, as an increase in peak MJCF has been recently linked to medial tibiofemoral cartilage loss and knee osteoarthritis progression. Measuring MJCF for a high-risk group, women, in real time and clinical settings would be useful in furthering knee osteoarthritis research, but measurement of this risk factor is typically restricted to a gait analysis lab setting. Therefore, a prediction model for MJCF was developed during walking and running for young female individuals. This model utilized the lab's previously developed instrumented insoles and two CNN models for task specific walking and running models. The models estimated the reduction modeling calculated MJCF with overall strong agreement (r>0.98). MJCF was predicted across the entire stance phase with strong correlation coefficients (r>0.98) for the walking and running models, as the results are comparable to previously developed wearable systems and neural networks used to estimate total knee joint forces. The walking results are also within the smallest detectable difference in peak MJCF, 0.25 BW, and relatively close to the smallest detectable difference for the running results. The greater MSE in running as compared to walking may be due to the greater variation in strike patterns during running. Walking typically begins with heel contact, while with running, especially at varying speeds, initial contact can range from the heel to toe. For both walking and running, the peak predictions are further from the reference values at larger MJCF. There are also fewer peak values at larger MJCF, and faster speeds are associated with larger tibiofemoral joint contact forces.

Knee joint contact force has been successfully predicted using neural networks and gait analysis data. Burton and colleagues predicted total knee joint contact force using participant height, weight, joint angles, moments, and ground reaction forces as inputs into neural networks. With their highest performing model, recurrent neural networks, they predicted knee contact force with similar correlation coefficients (r=0.94±0.4). Burton and colleagues had a more diverse sample size of 70 total knee replacement patients ranging in age from 45 to 80 years. Similar accuracies were achieved with a reduced number and type of model inputs, but also with a less diverse and smaller sample. To increase the model generalizability, dropout layers and early stopping were introduced. By using a wearable insole as inputs to the model rather than gait lab data, a system was created that could be used outside of a lab.

Using a convolutional neural network across 63 participants, some with knee osteoarthritis, they estimated MJCF with moderate correlation coefficients (r=0.82). This work with force plates shows the ability to reduce the number of inputs to the deep learning models and the potential for insole-based measurements, even in the absence of the kinematic data necessary for calculation of contact forces using musculoskeletal modeling. Using an instrumented insole rather than force plate data, greater correlation coefficients were achieved.

One other group has also estimated total knee joint contact force using just wearable sensors and neural networks. Stetter and colleagues estimated knee joint contact forces using inertial measurement units placed above and below the knee joint and an artificial neural network with two layers using leave-one-subject-out-cross-validation. On average they estimated the vertical compressive knee joint force with strong correlation coefficients for running (r=0.92±0.38) and walking (r=0.87±0.32). By creating task-specific models, separate models for walking and running, a more accurate estimate MJCF was achieved. The methods described herein also differ in the application of CNN, rather than feed-forward artificial neural networks, which include convolutional layers. CNN have shown greater success than feed-forward neural networks in previous research with the instrumented insoles and other previous research using force plates. The present tasks (walking and running) could likely be distinguished for example by step rate, or by the present/absence of double-support if using bilateral sensors.

To minimize the limitations of the relatively small sample size, leave-one-subject-out cross-validation was applied, which is a technique recommended to train a more generalizable model. The results may be generalizable to healthy young female individuals. An unequal amount of data to the walking and running models was also applied due to the small sample size. Running models had 105 stance cycles compared to 104 stance cycles for the walking model, while the walking model contained data from an extra participant. Collecting a larger amount of data and applying equal-sized data sets to the walking and running models would allow for a more just comparison of the two.

In the foregoing specification, implementations of the disclosure have been described with reference to specific example implementations thereof. It will be evident that various modifications may be made thereto without departing from the broader spirit and scope of implementations of the disclosure as set forth in the following claims. The specification and drawings are. accordingly, to be regarded in an illustrative sense rather than a restrictive sense.

What is claimed is:

1. A method for training a neural network model to determine medial joint contact force, the method comprising:
obtaining a plurality of training data comprising sets of corresponding force plate data, insole sensor data, and motion capture data, wherein the force plate data, insole sensor data, and motion capture data of each set was acquired simultaneously from a monitored gait;
extracting a plurality of heel strike to toe-off time periods from the plurality of training data;
assigning a gait categorization label corresponding to the plurality of training data;
determining a ground reaction force, a plurality of knee moments, and a plurality of knee angles based on said force plate data and motion capture data;
determining a plurality of joint contact forces at a plurality of time points during the plurality of heel strike to toe-off time periods;
providing an input training array to the neural network, the input training array comprising insole sensor data corresponding to the plurality of heel strike to toe-off time periods;
providing an output training array to the neural network, the output training array comprising the plurality of joint contact forces corresponding to the plurality of heel strike to toe-off periods;
training the neural network model to output a predicted joint contact force value corresponding to a calculated joint contact force measurement from force plate data, based only on insole data; and
storing the trained neural network model after it has been validated.

2. The method of claim 1, wherein said force plate data and said motion capture data were acquired using a force plate sensor and motion capture sensor directed to a subject who was also wearing one or more insole sensors.

3. The method of claim 1, further comprising determining 101 time points representing portions of a stance phase based on the plurality of training data.

4. The method of claim 1, wherein the output training array further comprises a cumulative load during a heel to toe-off period.

5. The method of claim 1, wherein the output training array further comprises a maximum force during a heel to toe-off period.

6. The method of claim 1, wherein the insole sensor data was acquired using an insole comprising more than three force sensors.

7. The method of claim 6, wherein the more than three sensors measure in three axes.

8. The method of claim 1, wherein the force plate data was acquired using a force plate installed in view of a camera acquiring the motion data.

9. The method of claim 1, wherein the plurality of heel strike to toe-off time periods are extracted by comparing the plurality of training data to one or more threshold values.

10. The method of claim 1, further comprising identifying a plurality of origins and midpoints in said motion capture data.

11. A system for determining medial joint contact force of a user, the system comprising:
an insole, the insole comprising: at least one insole sensor configured to measure a force associated with a subject's gait, and a wireless transmitter connected to the at least one insole sensor to transmit output data of the at least one insole sensor;
a processor in communication with the insole;
a memory in communication with the processor and having instructions stored thereon that, when executed, cause the processor to:
receive a plurality of output data from the at least one insole sensor;
determine an activity category corresponding to the plurality of output data;
extract a temporal bin of the data, associated with a heel strike to toe-off period;
provide the temporal bin of data and the corresponding activity category to a trained neural network model;
determine, using the trained neural network model, a medial joint contact force occurring during the heel strike to toe-off period;
generate a joint force summary; and
transmit the joint force summary to the user.

12. The system of claim 11, wherein the activity category is at least one of: a running gait or a walking gait.

13. The system of claim 11, wherein the memory further has instructions stored thereon that, when executed, cause the processor to:
generate a recommendation corresponding to one or more recommended changes to a trunk lean or a toe progression angle of the user.

14. The system of claim 11, wherein the memory further has instructions stored thereon that, when executed, cause the processor to:
transmit the joint force summary to a medical professional.

15. The system of claim 11, wherein the plurality of output data comprises a plurality of steps performed by the user.

16. The system of claim 11, wherein the trained neural network model is selected from a plurality of neural network models corresponding to a plurality of activity categories.

17. The system of claim 11, wherein the memory further has instructions stored thereon that, when executed, cause the processor to:
compare the medial joint contact force to a user-defined force value; and
send an alert to a user device based on the comparison.

18. The system of claim 11, wherein the processor and memory are disposed in a mobile device associated with the subject, and the at least one insole sensor comprises a wireless transmitter to transmit data from the at least one insole sensor to the mobile device for real time processing.

19. The system of claim 18, wherein the insole comprises at least one sensor in a heel location of the insole, at least one sensor in a metatarsal region of the insole, and at least one sensor in a toe region of the insole, and further wherein the memory comprises instructions that, when executed, cause the processor to:
initiate a measurement phase based on user input;
determine whether the subject is walking or running in real time based on at least one of current location data from the mobile device or current frequency of changes in data from the at least one sensor in the metatarsal region;
determine a duration of the temporal bin based in part upon whether the subject is walking or running; and
display a notification to a screen of the mobile device reflecting at least one of peak joint force per foot during the measurement phase, average joint force per foot during the measurement phase, current joint force per foot, and gait modification recommendations.
